(19) United States
(12) Patent Application Publication
(10) Pub. No.: US 2004/0030263 A1
(43) Pub. Date: Feb. 12, 2004

(54) Undamaged tissue collection assembly and method

(75) Inventors: William R. Dubrul, Redwood City, CA (US); Eric L. Buehlmann, Redwood City, CA (US); Jeffrey A. Smith, Santa Rosa, CA (US); John A. Scholl, Danville, CA (US); Barbara A. Hird, Berkeley, CA (US)

Correspondence Address: O'MELVENY & MEYERS, 114 PACIFICA, SUITE 100, IRVINE, CA, 92618, US

(73) Assignee: Artemis Medical, Inc., Hayward, CA

(21) Appl. No.: 10/229,900

(22) Filed: Aug. 28, 2002

Related U.S. Application Data
(60) Provisional application No. 60/315,913, filed on Aug. 29, 2001.

(51) Int. Cl. A61B 10/00

(52) U.S. Cl. 600/565

(57) ABSTRACT

A tissue sample collection assembly collects undamaged cells from a tissue specimen comprising a damaged tissue layer at a margin thereof. The assembly includes a tissue-severing device used to separate at least a portion of the damaged tissue layer. The tissue-severing device may have a tissue-adhesive surface so that undamaged cells may contact and adhere to the tissue-adhesive surface for subsequent analysis. The assembly may comprise an apertured device comprising inner and outer surfaces with apertures passing therebetween. The apertures may be sized and shaped so that when the inner surface of the apertured device is pressed against a tissue specimen, portions of an undamaged tissue layer of the tissue specimen pass through the apertures and past the outer surface so that the tissue-severing device can sever the undamaged tissue layer portions from the remainder of the tissue specimen. Exposed undamaged tissue may adhere to a tissue-adhesive surface of the tissue-severing device.

### CROSS-REFERENCE TO RELATED APPLICATION

This application claims the benefit of provisional patent application No. 60/315,913 filed on Aug. 29, 2001 and entitled Diagnostic Apparatuses And Methods For Use.

### BACKGROUND OF THE INVENTION

This invention relates to medical devices and methods, in particular apparatus and methods for the collection of undamaged tissue from a tissue specimen of the type having damaged tissue at the margin of the tissue specimen.

To be able to analyze tissue intraoperatively, immediately postoperatively as well as postoperatively is extremely advantageous to the physician. This tissue analysis is often necessary for definitive surgical planning or other postoperative therapy or care.

Cancer results in over 1,500 deaths every day in the U.S. (550,000 every year). Therapy modalities for cancer are plentiful and continued to be researched with vigor. Still, the preferred treatment continues to be physical removal of the cancer. When applicable, surgical removal is preferred (breast, colon, brain, lung, kidney, etc.). Open, excisional, surgical removal is often extremely invasive and efforts to remove cancerous tissue in a less invasive way continue, but have not yet been perfected.

Still, the only cure for cancer continues to be early diagnosis and subsequent early treatment. As cancer therapies continue at an earlier stage of diagnosis, the cancerous tissue is smaller and smaller. Early removal of these smaller cancers demands new techniques for removal and obliteration that are less invasive than present techniques. Patent applications and patents assigned to Artemis Medical, Inc., as well as other technologies of such companies as SenoRx, Vivant Medical, NeoThermia, Sanarus, Calypso Medical, USSC, etc., describe percutaneous, potentially less traumatic tissue removal techniques. In the case of tissue removal from the breast (but this is often true for other tissue such as the variety of different types of sinuous tissue often found in plaque build up in arteries in the case of occlusive vascular disease), the tissue being removed is often difficult to cut and hence difficult to remove. See, for example, U.S. Pat. Nos. 6,270,464; 6,179,860; and 6,221,006; and International Publication No. WO 00/74561.

There are many techniques available today, such as the USSC ABBI and the Site Select from Imagine Corporation that attempt to accomplish this but with results that leave the breast physician desiring. The Mammotome from J&J and the MIBB from USSC also require large bore access to accomplish biopsy but only remove slivers of tissue. Further, as reported at the Mar. 13, 2000 symposium of the American Society of Surgical Oncologists (SSO) in New Orleans, Stereotactic Core Biopsy (SCB) such as those accomplished with the BARD TRU-CUT, Mammotome or MIBB (the Mammotome and MIBB are referred to as ‘vacuum assisted’ biopsy devices) fall short in providing definitive answers to detail precise surgical regimens after this SCB type biopsy, especially with DCIS (Ductal Carcinoma In Situ), LCIS (Lobular Carcinoma In Situ), ADH (Atypical Ductal Hyperplasia) and other discordant findings usually of early detected cancers or pre-cancerous lesions. In this study presented by Dr. Ollila et al from the University of North Carolina, Chapel Hill, the investigators discovered that there is evidence that histology and pathology is compromised due to the damage that is done by these conventional techniques. Hence for many reasons, the least of which is not that DCIS, LCIS and ADH are becoming more detectable and hence more prevalent in breast cancer diagnosis in the U.S., there is a growing need to improve upon these core (BARD TRU-CUT and others (Medi-Tech, Cook, MD Tech, etc.)), vacuum assisted core biopsy systems (Mammotome or MIBB) as well as the established large core biopsy systems (Site Select and ABBI). Hence these improved systems that realize a large, contiguous piece of tissue through a smaller puncture site as described above (Artemis, Vivant, SenoRx, NeoThermia, Sanarus, Calypso, etc.) yield many of the preferred characteristics that the physician needs and wants. However there are always trade-offs. As far as the inventors know, all of the aforementioned technologies that remove a large contiguous piece of tissue through a smaller cannula, as well as the ABBI that uses a large coring (20-40 mm) technology from the skin all the way to the suspect lesion (or cancer), utilize an external energy source to ‘sever’ or cut the tissue for removal because of the odd characteristics of breast tissue. The aforementioned external energy sources include, but are not limited to RF (Radio Frequency), electrocut or electrocautery, cryosurgical, mechanical (i.e. vibrational, ultrasonic, etc.), laser, etc.

The trade-off with using these external energy sources is the fact that they tend to modify or damage tissue characteristics that may be detrimental to accurate or definitive tissue analysis. In the case of breast cancer this damaged tissue is detrimental to doing definitive pathological analysis of the cancer or suspect lesion. This analysis is often realized in a procedure known as ‘conization’. Conization is used in a procedure often referred to as a loop procedure. When a woman is presented with an atypicical pap smear of the cervix, the OB/GYN or other physician often refers her to this procedure. The physician usually uses an ‘electrocut loop’ device to trim off a small piece of the cervix for analysis. The energized loop often leaves what is referred to as an ‘electrocautery artifact’, that is a change of the tissue due to the electrical energy generated. The pathologist usually takes the removed cervical tissue sample and begins to analyze the tissue by cutting back from the area with the artifact. He/she removes small layers of tissue until reaching non-affected tissue (i.e. tissue with no electrocautery artifact). Upon reaching that tissue, if tissue is found with normal cells (not atypical cells), the patient is told that the conization was successful and no additional treatment/diagnosis/care is necessary until the next scheduled pap smear. If the pathologist finds atypical (not normal) cells then the patient is required to come back a few (or several) days later for additional conization or other treatment.

In the case of breast cancer surgery, the patient is usually on the operating room table. The surgeon removes the cancerous tissue, hoping to ‘get it all’ by removing the cancer and healthy tissue known as ‘clean’ margins. There is significant controversy as to what defines ‘clean’ margins. Some physicians call ‘clean’ margins as one cell of ‘normal’ tissue between the cancerous tissue and the external surface of the removed tissue. Other physicians prefer to define clean margins as 10 mm of tissue between the cancer and the healthy tissue. Regardless of the controversy that exists on what constitutes clean margins, all physicians agree that they must know what the margin distance is. Certainly other criteria exist, but if margins are poor or good determines how the patient is followed. For example if no margins exist, most often additional tissue is removed via an additional surgical procedure. If margins are only fair, the physician may prefer to use an adjunctive therapy that is aggressive with no additional surgery. If margins are good, usually the physician will still prescribe additional adjunctive therapy, but not necessarily as aggressive as with only fair margins.

Hence with the importance put upon margins, it would be extremely beneficial for the surgeon/physician to know if clean margins exist at the time of surgery. For example if they were to know, at the time of surgery, with the patient still on the operating room table, that there are bad margins (AKA ‘dirty margins’), the surgeon could re-enter the cavity where the cancerous tissue was removed and then remove additional tissue. This would allow the patient to have fewer surgeries and thus fewer potentially deleterious effects that accompany every surgical procedure. Such a deleterious effect could be as severe as death.

Because of this important need of determining margins at the time of surgery, a technique known as ‘Touch Prep’ has been developed. Touch Prep refers to a preparation of the cancerous tissue that is removed and an immediate analysis of that tissue intraoperatively or immediately post operatively. Touch Prep can basically described as follows.

Upon removal of the tissue from the patient, the surgeon marks the orientation of the tissue with respect to how it was removed from the body. This marking convention is different at each institution, but is usually done with the placement of sutures on the tissue removed. For example, the surgeon may place one suture on the anterior side; two sutures on the posterior side, three sutures on the medial side and the lateral side would be defined with no sutures.

The tissue is then immediately sent to pathology where it is stained with various colors again using a convention in the particular institution that would indicate the orientation of how the tissue was removed from the patient (i.e. anterior, posterior, medial, lateral, etc.). This color staining allows the pathologist and the surgeon to communicate as to the condition of the margins on the tissue. The staining does not affect pathological analysis except for the fact that the pathologist knows which side of the tissue he/she is analyzing.

At this point, the pathologist takes the tissue and smears tissue samples from the different colors onto ‘analyzing slides’ for immediate microscopic evaluation. As the pathologist is searching each individually ‘colored’ slide, he/she knows the particular orientation of where these cells came from in the patient. If the pathologist sees irregular or cancerous cells on a particular slide, he/she can then immediately notify the surgeon that there exist irregular margins on a particular side of the tissue sample removed.

Once ‘irregular margins’ is communicated to the surgeon, re-intervention is usually accomplished. Because the surgeon knows that irregular margins exist on a particular side of the tissue removed, they are able to re-enter the cavity and remove additional tissue from the patient in that particular area.

At that point, often, the Touch Prep is repeated again until ‘clean margins’ have been determined. This procedure allows the patient to usually have only one surgical intervention as opposed to doing analysis on the tissue post operatively and then having subsequent pathology only after the patient has left the operating room and thus requiring an additional surgical intervention with the accompanying risks.

This new Touch Prep type of analysis is not completely perfect, but is becoming more commonplace due to the obvious advantages that come with it. However, with the addition of the new technologies described above, those being removal of tissue with external energy sources, the Touch Prep procedure can be compromised. If electro-cautery artifacts (or any type of damaged tissue for that matter) exist on the external sample of the tissue removed, this Touch Prep may be compromised.

Further, many physicians are now beginning to use such a modification of the Touch Prep procedure at the time of biopsy so that determination of the biopsy sample is known immediately at the time of diagnosis during biopsy. For example, some physicians using a ‘core’ type technology may smear the cored tissue sample onto a pathology slide. The slide is then immediately taken to pathology so that a determination of the tissue can be at least partially accomplished immediately. The coloring or other orientation of the biopsy sample may or may not be done. In the case where it is not done, the orientation is not necessarily accomplished, but only whether cancer or other irregularity exists. In either case it allows the physician immediate determination of whether irregular cells exist instead of waiting the usual day or more for said analysis.

### SUMMARY OF THE INVENTION

The present invention relates to the analysis of undamaged tissue from a tissue specimen in which some of the tissue at the margin of the tissue sample may have been damaged during removal procedures, such as electrocautery (RF or other electrosurgical apparatus), mechanical cutting using wire and scalpel-like devices, vibrational devices, cryogenic procedures, thermal procedures, and compression techniques.

A first aspect of the invention is directed to a tissue sample collection assembly, for collecting undamaged cells from a tissue specimen, comprising a reference surface placeable against a tissue specimen, the tissue specimen comprising a damaged tissue layer at a margin of the tissue specimen, the damaged tissue layer having a first, expected thickness. The assembly also includes a tissue-severing device spaced apart from the reference surface by a second distance, the second distance being at most about 40% greater than the first thickness. The tissue-severing device may be used to separate at least a portion of the damaged tissue layer of the tissue specimen so that undamaged cells may be analyzed. The tissue-severing device may have a tissue-adhesive surface facing the reference surface so that undamaged cells may contact and adhere to the tissue-adhesive surface. The tissue-severing device and the reference surface may be fixed or movable relative to one another.

A second aspect of the invention is directed to a tissue sample collection assembly, for collecting undamaged cells from a tissue specimen, comprising a reference surface placeable against a tissue specimen, the tissue specimen comprising a damaged tissue layer at a margin of the tissue specimen, the damaged tissue layer having a first, expected thickness. The assembly also includes a tissue-severing device spaced apart from the reference surface by a second distance, said second distance chosen to be at least as great as the first thickness. The tissue-severing device has a tissue-adhesive surface facing the reference surface. The tissue-severing device may be used to separate at least a portion of the damaged tissue layer of the tissue specimen so that undamaged cells may contact and adhere to the tissue-adhesive surface. The tissue-severing device and the reference surface may be fixed or movable relative to one another.

A third aspect of the invention is directed to a tissue sample collection assembly, for collecting undamaged cells from beneath a damaged tissue layer at a margin of a tissue specimen, the damaged tissue layer having a first thickness. The assembly comprises an apertured device comprising inner and outer surfaces with apertures passing therebetween and a tissue-severing device having a tissue-adhesive surface placeable adjacent to the outer surface. The apertures are sized and shaped so that when the inner surface of the apertured device is pressed against a tissue specimen, portions of an undamaged tissue layer of the tissue specimen pass through the apertures and past the outer surface so that the tissue-severing device can sever the undamaged tissue layer portions from the remainder of the tissue specimen. Exposed undamaged tissue may adhere to the tissue-adhesive surface of the tissue-severing device.

A fourth aspect of the invention is directed to method for analyzing undamaged tissue from a margin of a tissue specimen of a type in which the margin comprises a damaged tissue layer overlying undamaged tissue, said damaged tissue layer comprising damaged tissue. The expected thickness of the damaged tissue is determined. The thickness of the damaged tissue to be removed is chosen, the chosen thickness being greater than the expected thickness. At least a portion of the damaged tissue layer is removed to expose a region of the undamaged tissue. Tissue from at least a chosen one of the undamaged tissue region and the inner surface of the portion of the damaged tissue region is analyzed. The chosen thickness is minimized to help ensure that the tissue analyzed is close to the margin of the tissue specimen to help with the determination of whether there is tissue of interest at the margin. The removing step may comprise pressing an apertured device and the damaged tissue layer against one another causing damaged tissue to protrude through one or more apertures, and separating at least some of the protruding damaged tissue from the remainder of the tissue specimen to expose the region of undamaged tissue. The separating step may comprise passing a blade over the apertured device. The apertured device may comprise a generally tubular braided device. The removing step may also comprise passing a tissue-separating tool over the damaged tissue layer; the passing step may comprise adhering tissue from the undamaged tissue region to a tissue sample region of the tissue-separating tool.

A fifth aspect of the invention is directed to method for analyzing undamaged tissue from a margin of a tissue specimen of a type in which the margin comprises a damaged tissue layer overlying undamaged tissue, said damaged tissue layer comprising damaged tissue. A tissue sample is removed from the margin of the tissue specimen, the tissue sample comprising damaged tissue and undamaged tissue. An analysis technique that differentiates between damaged tissue and undamaged tissue in a tissue sample is chosen. Undamaged tissue from the tissue sample is analyzed. The analyzing step may be carried out using, for example, at least one of magnetic resonance imaging (MRI), ultrasound (US), positron emission tomography (PET), computed tomography (CT), X-ray, photo-spectral analysis and electron microscopic analysis.

A sixth aspect of the invention is directed to method for analyzing undamaged tissue from a margin of a tissue specimen of a type in which the margin comprises a damaged tissue layer overlying undamaged tissue, said damaged tissue layer comprising damaged tissue. A tissue sampling device is inserted into the damaged tissue layer and through damaged tissue to access undamaged tissue. An undamaged tissue sample is captured using the tissue sampling device. The undamaged tissue sample is removed from the tissue specimen. The undamaged tissue sample is analyzed.

A seventh aspect of the invention is directed to method for analyzing undamaged tissue from a margin of a tissue specimen of a type in which the margin comprises a damaged tissue layer overlying undamaged tissue, said damaged tissue layer comprising damaged tissue. A tissue characteristic analysis probe is inserted into the damaged tissue layer and through damaged tissue to access undamaged tissue. The undamaged tissue is analyzed for said tissue characteristic using the tissue characteristic analysis probe.

An eighth aspect of the invention is directed to method for obtaining a tissue analysis sample following removal of a tissue specimen through an access track of a patient, the access track opening into a tissue specimen excision void of the patient. A sample retrieval structure, movable between a collapsed state an expanded state, is selected. The sample retrieval structure has a tissue-adhesive surface. The sample retrieval structure, in a collapsed state, is inserted along an access track and into an excision void of a patient. The sample retrieval structure is expanded to an expanded state. The tissue-adhesive surface is pressed against a wall defining the excision void thereby causing tissue from said wall to adhere to the tissue-adhesive surface. The sample retrieval structure is collapsed to a collapsed state. The sample retrieval structure, together with tissue adhering to the tissue-adhesive surface, is removed from the patient, whereby said tissue adhering to the tissue-adhesive surface may be analyzed.

Various advantages may arise from the various aspects of the invention. For example, one advantage may be the controlled removal of the damaged tissue layer from the tissue specimen so that Touch Prep can still be accomplished. Another advantage is that the tissue just beneath the damaged tissue can be analyzed so that margin and/or tissue quality can be determined. The invention may be carried out in a safe and cost-effective manner. The invention may also be made to be simple to use and in a very real sense simple to understand. This will encourage its adoption and use by medical personnel. The invention generally relates to procedures with which the medical profession is familiar so that the skills that have been learned from previous experience will continue to have applicability.

Other features and advantages of the invention will appear from the following description in which preferred embodiments have been discussed and detail in conjunction with the accompanying drawings.

### BRIEF DESCRIPTION OF THE DRAWINGS

FIG. 1 illustrates a conventional tissue specimen after removal from the patient's body, the tissue specimen having orientation sutures placed thereon by the surgeon;

FIG. 2 illustrate the removal of a portion of damaged tissue from the margin of a tissue specimen by a skiving tool;

FIG. 3 is a front elevational view of the skiving tool of FIG. 2;

FIG. 4 is an enlarged perspective view illustrating the removable double-adhesive-sided tape on the blade of the skiving tool of FIG. 3;

FIG. 5 is a front elevational view of a multiple bladed skiving tool;

FIG. 6 is a simplified cross-sectional view of a sample collection assembly made according to the invention, shown adjacent the tissue specimen, having a blade offset from a reference surface for the removal of a predetermined thickness of tissue from the tissue specimen;

FIG. 7 illustrates an alternative embodiment of the sample collection assembly of FIG. 6 in which the reference surface is a vacuum surface and the blade can move relative to the reference surface;

FIG. 8 illustrates a stiff, single aperture device being pressed against a tissue specimen prior to moving a blade across the single aperture device to slice off protruding damaged tissue from the margin of the tissue specimen so to expose undamaged tissue;

FIG. 9 illustrates an alternative embodiment of the device of FIG. 8 including a stiff, multiple aperture device used in conjunction with a blade;

FIGS. 10 and 11 illustrate a further alternative embodiment in which a motorized skiving tool includes an apertured mesh, which is pressed against a tissue specimen, and a rotatable blade, which slices protruding damaged tissue from a tissue specimen;

FIG. 12 shows the tissue specimen of FIG. 11 after the protruding damaged tissue has been sliced away leaving exposed undamaged tissue regions;

FIG. 13 illustrates a generally tubular braided device and a tissue specimen to be placed into the generally tubular braided device;

FIG. 14 illustrates the generally tubular braided device of FIG. 13 with the tissue specimen therein, the braided device having been placed in tension causing tissue to protrude through the apertures of the braided device to permit a blade or other tissue separating or removing device to cut or slice off or otherwise remove the protruding damaged tissue;

FIG. 14A is an enlarged view of a portion of FIG. 14 illustrating the tissue protruding through the apertures formed between the fibers of the braided device;

FIG. 15 shows a specimen capturing and removal device having a tissue specimen captured within the interior of a two layer braided device;

FIG. 16 is a simplified enlarged cross-sectional view taken a long line 16-16 of FIG. 15 showing an outer, tissue-impervious tubular braided device connected to and extending from an inner, tubular braided device with apertures, the inner, tubular braided device being similar to the braided device of FIGS. 13 and 14;

FIG. 17 shows the device of FIG. 15 after the outer braided device has been separated from the placement shaft and pulled back over the inner braided device so that placing the braided device in tension causes damaged tissue to protrude out through the apertures formed between the fibers of the inner braided device in a manner similar to the embodiment of FIG. 14;

FIGS. 18 and 19 illustrate a closed end braided device containing a tissue specimen and a generally cylindrical blade moving over the exterior of the braided device causing protruding damaged tissue from the margin of the tissue specimen to be sliced off or severed so to expose undamaged tissue;

FIG. 20 illustrates a coiled, generally cylindrical blade which permits the transverse dimensions of the blade to be adjusted according to the transverse dimension of the tissue specimen within the braided device of FIGS. 18 and 19;

FIG. 21 shows a tissue specimen being placed in an open-ended container;

FIG. 22 shows the specimen of FIG. 21 housed within the container and a cutting device being driven into the container;

FIG. 23 illustrates a specimen of FIG. 22 after the cutting device has sliced away a major portion of the damaged tissue layer lying against the inner wall of the container;

FIGS. 24-26A illustrate obtaining undamaged tissue from a cavity using a void wall tissue sample collection assembly comprising a radially expandable and contractible apertured void wall engagement device and a blade movable along the inner surface of the void wall engagement device to remove tissue protruding through the apertures thereof to expose undamaged tissue regions;

FIG. 27 illustrates a needle type tissue removal technology (not dissimilar to FNA (Fine Needle Aspiration)) where a stop on the device is used so that tissue cell sampling can be precisely taken just below the damaged external tissue layer; and

FIG. 28 illustrates two tissue characteristic analysis devices, one having multiple tissue characteristic analysis probes and the other having a single tissue characteristic analysis probe, each having a stop so that the probes may be accurately placed to a certain depth just below the damaged tissue layer for the analysis of one or more tissue characteristics.

### DETAILED DESCRIPTION OF THE INVENTION

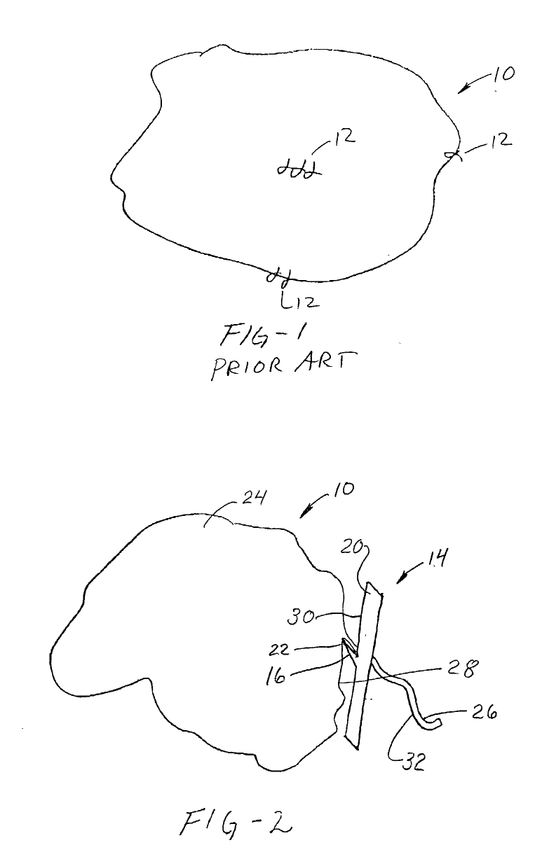

FIG. 1 is an illustration of a conventional tissue specimen 10 that has been removed from the body in a condition that it can be analyzed from a histological/pathological perspective immediately upon removal so that should additional tissue removal or other therapy be required then immediately after analysis of the tissue, this can be accomplished. A variety of analytic tools could be used to determine tissue characteristics. These tools include, but are not limited to the use of a computerized fiber-optic and/or laser technology, microscopic spectrometer to analyze protein density, flow cytometry, instant mitotic index, or instant immunohistochemistry to assess whether cells on the margin of the specimen are malignant or not would determine if the excised specimen contained all of the tumor or if some had been left behind and/or if sufficient margins exist, FNA (fine needle aspiration), skiving of the tissue for Touch Prep analysis, impedance testing of the tissue, resistance or other electrical measurement, density, reflectivity, refractivity, etc. In FIG. 1, you can see 1, 2 and then 3 orientation sutures 12 that have been placed by the surgeon to orient the tissue with the patient's body.

FIGS. 2, 3 and 4 illustrate a first embodiment of a tissue sample collection assembly 14, in particular a skiving (tissue-separating) tool 14, comprising a blade 16 positioned adjacent to an opening 18 formed in the body 20 of skiving tool 14. Blade 16 has a tissue-severing edge 22. Tissue specimen 10 has a margin 24 comprising a layer of tissue that may have been iatrogenically damaged due to energy used to remove tissue specimen 10 originally. Skiving tool 14 is used to remove a portion 26 of the damaged tissue layer to expose an undamaged tissue region 28. It should be noted that the thickness of portion 26 of the damaged tissue layer is chosen according to the expected thickness of the damaged tissue. In some cases the thickness of the damaged tissue will only be about 0.20 mm. The thickness of portion 26 can be controlled by, for example, controlling the compression force exerted on tissue sample 10 and the distance between edge 22 and a reference surface 30 of body 20 of skiving tool 14.

It will usually be desired to choose the thickness of portion 26 to be as small as possible while still exposing undamaged tissue region 28. This will help to ensure that the undamaged tissue analyzed is as close to margin 24 of tissue specimen 10 has possible to help with the determination of whether there are cancer, or other cells of interest, at the margin. Therefore, it is preferred that the thickness of portion 26 be about 40% greater, and preferably about 20% greater, and most preferably about 10% greater than the expected thickness of the damaged tissue layer. Also, it should be understood that undamaged tissue can be obtained from undamaged tissue region 28 as well as the underside 32 of portion 26. Skiving tool 14 may be designed to allow for the small incremental removal of damaged tissue rather than removal of the entire damaged tissue layer in a single pass.

The embodiment of FIGS. 2-4 obtains undamaged tissue from underside 32 of portion 26 by the use of a removable, double-adhesive-sided tape 34 on the inner surface 38 of blade 16, which passes against underside 32. After removal of portion 26, tape 34 is removed, as suggested in FIG. 4, and the undamaged tissue adhering to adhesive service 36 may be marked, indicating where on tissue specimen 10 the undamaged tissue was obtained from, and then analyzed. Adhesive surface 36 may be provided in other manners. For example, inner surface 38 may be made so that undamaged tissue adheres directly to surface 38; this may be achieved by, for example, providing surface 38 with a suitable texture or roughness to collect tissue or coating surface 38 with a suitable adhesive. Also, blade 16 may be removable from body 20 to facilitate tissue analysis. Blade 16 may also be made to be of a translucent or transparent material to facilitate visual inspection of the removed tissue without the need to transfer the remove tissue from blade 16 to, for example, an analyzing slide. Additional embodiments and aspects of the invention are discussed below with like reference numerals referring to like elements.

FIG. 5 illustrates a multiple bladed skiving tool 40. Tool 40 is similar to tool 14 but has a number of curved blades 42, each blade having a curved edge 44.

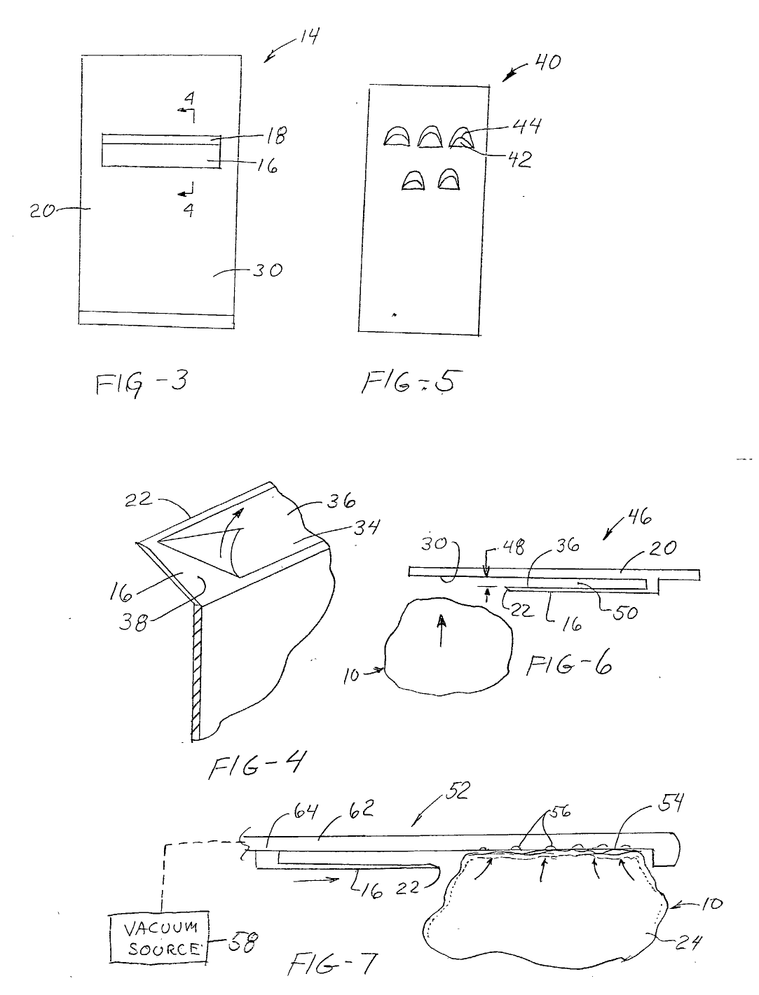

FIG. 6 illustrates another embodiment of a sample collection assembly 46 in which a blade 16 is used as the tissue-severing device, blade 16 being affixed to body 20 and oriented parallel to reference surface 30. Blade 16 is spaced apart from reference surface 30 by an offset 48, offset 48 being chosen to be at least slightly greater than the expected thickness of the damaged tissue. A portion 26 of tissue specimen 10 is separated from the remainder of the tissue specimen and is captured within the space 50 defined between reference surface 30 and tissue-adhesive surface 36.

FIG. 7 illustrates a further embodiment of a sample collection assembly 52 similar to the embodiment of FIG. 6. However, assembly 52 comprises a tissue contact surface 54 defining a number of vacuum ports 56, the vacuum ports coupled to a vacuum source 58. As suggested in FIG. 7, the margin 24 of specimen 10 is pulled into contact with surface 54 because of the suction forces created at vacuum ports 56. Assembly 52 also comprises a movable blade 60 connected to body 62 of assembly 52 by a slide coupling 64 to permit tissue at surface 54 to be cut or separated from the remainder of specimen 10. If desired, after the removal of the severed tissue at surface 54, the newly exposed tissue could be placed against surface 54 and blade 60 used to slice off a second tissue sample of undamaged cells for analysis.

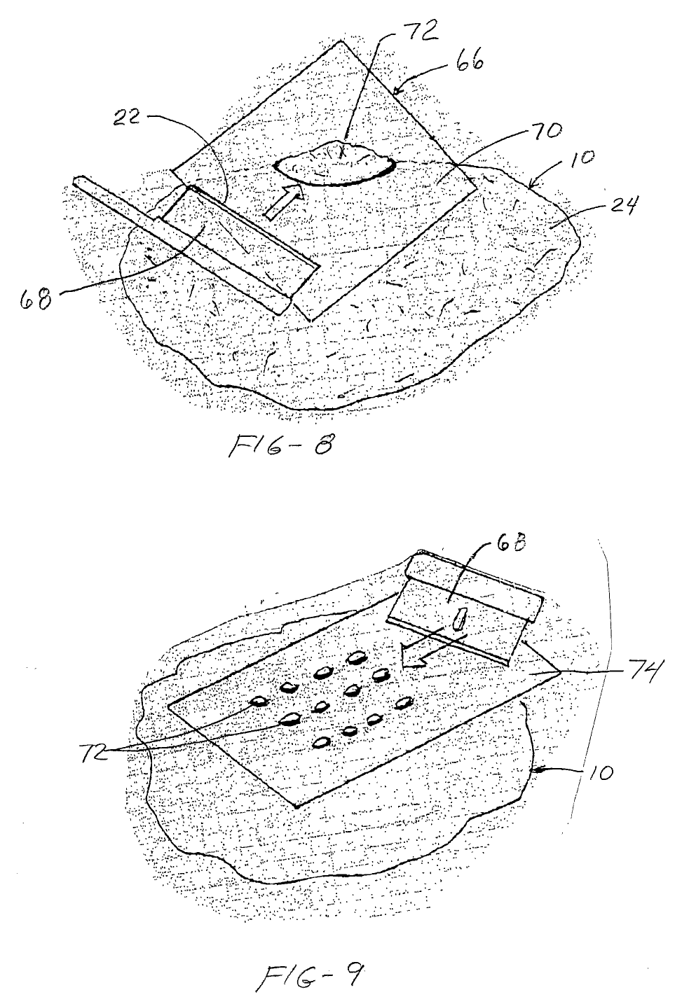

FIG. 8 illustrates a stiff, single aperture device 66 being pressed against a tissue specimen 10 prior to moving a blade 68 across outer surface 70 of device 66 to slice off protruding damaged tissue 72 from margin 24 of the tissue specimen so to expose undamaged tissue. As in the above embodiments, undamaged tissue may be collected, for example, from an adhesive surface on the underside of blade 68 or from the undamaged tissue region created by removing protruding damaged tissue 72. FIG. 9 illustrates an alternative to the embodiment of FIG. 8 comprising a stiff, multiple aperture device 74 which permits protruding damaged tissue 72 to be created at each aperture and then removed by blade 68. Alternatively, protruding damaged tissue 72 could be removed using sandpaper, a rasp or other suitable structures or techniques. The size of the apertures and the force exerted on tissue specimen 10 largely determines how much tissue is pushed up through the apertures.

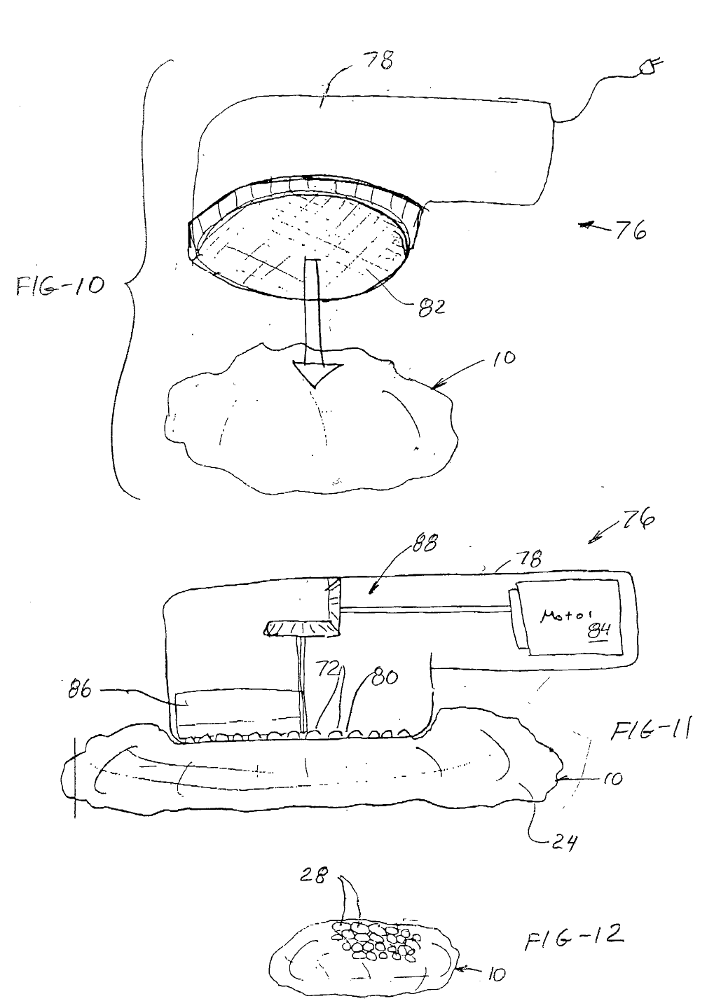

FIGS. 10 and 11 illustrates a further sample collection assembly 76 in the form of a motorized skiving tool 76, comprising a hand-held body 78 having an opening 80 covered by an apertured mesh 82. Tool 76 comprises a motor 84 connected to a rotatable blade 86 by a drivetrain 88. The apertured mesh 82 of tool 76 is pressed against margin 24 of tissue specimen 10 creating protruding damaged tissue 72 which is sliced off by the rotation of rotatable blade 86. This creates a plurality of undamaged tissue regions 28, shown in FIG. 12. Again, undamaged tissue may be collected, for example, from tool 76, such as from blade 86, or from undamaged tissue regions 28.

FIGS. 13, 14 and 14A illustrate a sample collection assembly 90 comprising a generally tubular braided device 92 and a blade 64. Tissue specimen 10 is placed into the interior of braided device 92 and the ends of device 92 are pulled to place device 92 into tension creating protruding damaged tissue 72 between the filaments or yarns 94 of device 92. Protruding damaged tissue 72 is then removed using, for example, blade 68.

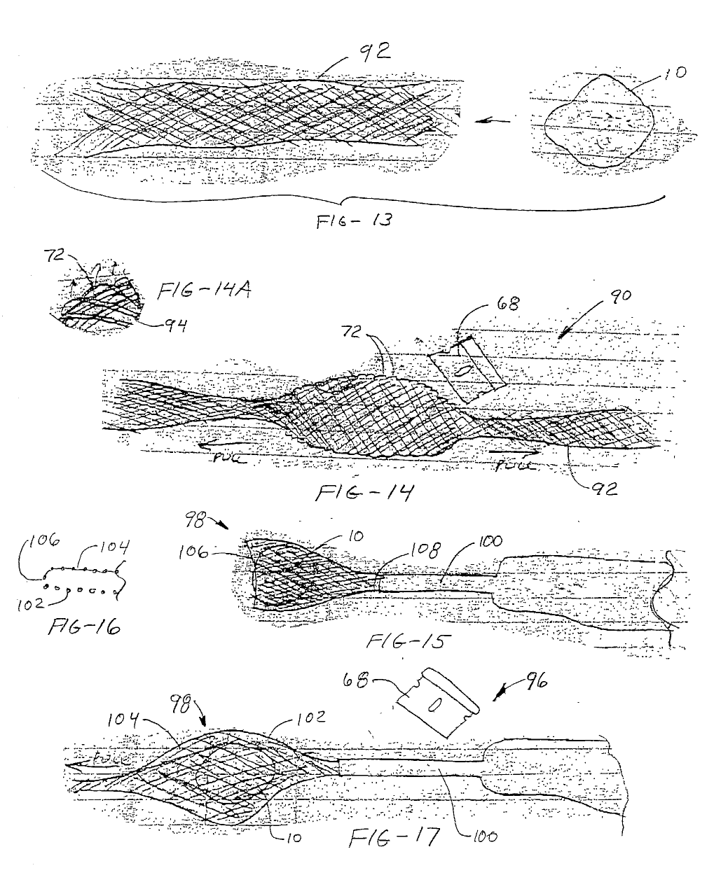

FIGS. 15, 16 and 17 and illustrates a sample collection assembly 96 comprising a specimen capturing and removal device 98 mounted to the distal end of a placement sheath 100. This structure may be similar to that described in U.S. Pat. No. 6,221,006. Device 98 comprises an inner, tubular braided device 102 with apertures, similar to braided device 92 of FIG. 13, connected to an outer, tissue-impervious covering 104 along their joint distal edge 106. Covering 104 is preferably a tubular braided device in which the apertures have been sealed so to prevent passage of tissue therethrough. A tissue specimen 10 is shown captured within device 98. The provision of outer, tissue-impervious covering 104 helps to prevent seeding of tissue from specimen 10 when sample collection assembly 96 is used to capture and extract tissue specimen 10 from a patient. After the tissue specimen has been retrieved from the patient, the proximal end 108 of outer covering 104 is cut or otherwise separated from placement sheath 100 and pulled distally back over inner braided device 102 to place device 98 in tension thus squeezing tissue specimen 10 in much the same manner as shown in FIGS. 14 and 14A. Protruding damaged tissue 72 may then be removed from inner braided device 102 to provide access to undamaged tissue as discussed above.

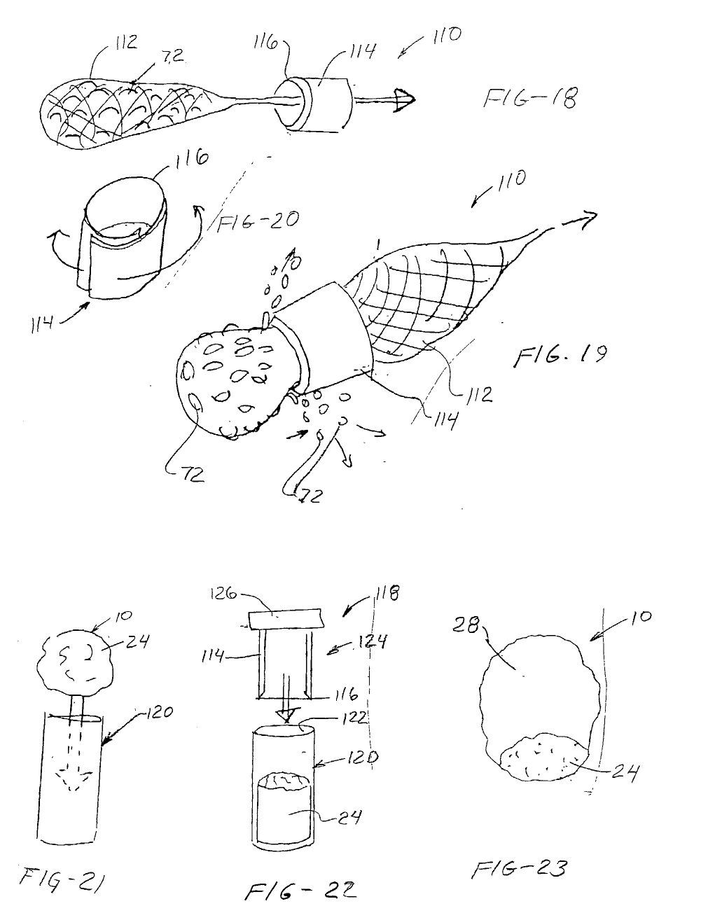

FIGS. 18, and 19 illustrate a sample collection assembly 110 comprising a closed-end braided device 112, within which a tissue specimen 10 is placed, and a generally cylindrical blade 114. Blade 114 has a substantially continuous loop, curved blade edge 116 and a transverse dimension sized to provide a desired constricting force on braided device 112 and tissue specimen 10 therein as blade 114 is passed over braided device 12 severing protruding damaged tissue 72 as suggested in FIG. 19. FIG. 20 illustrates blade 114 being a coiled, generally cylindrical blade in which the transverse dimension of the blade can be adjusted according to the transverse dimension of the particular tissue specimen 10. The resilience of blade 114 may be made such that it self-adjusts its diameter, over a range of diameters, according to the size of the tissue specimen.

FIGS. 21 and 22 illustrate a sample collection assembly 118 comprising a cylindrical, open ended container 120 within which a tissue specimen 10 is inserted. Container 120 is preferably a cylindrical container but may have other cross-sectional shapes as well. The inside diameter of container 120 is preferably chosen to be somewhat smaller than the transverse dimension of tissue specimen 10 so that margin 24 of tissue specimen 10 conforms to the inner wall 122 of container 120 as suggested in FIG. 22. Assembly 118 also includes a cutting device 124 comprising a cylindrical blade 114 extending from a handle 126. The diameter of blade 114 is chosen to be sufficiently less than the diameter of inner wall 122 so that when blade 114 is forced into container 120, the gap between blade 114 and inner wall 122 is sufficient so that the layer of tissue removed from tissue specimen 10 has the desired thickness. Appropriate centering structure, such as an outer tube extending from handle 126 which engages the outer surface of container 120, may be used. FIG. 23 illustrates tissue specimen 10 after removal of a portion of the damaged tissue layer from the margin of the tissue specimen to create a relatively large undamaged tissue region 28. As discussed above, undamaged tissue for analysis may be obtained from, for example, the inner or outer surfaces of blade 114, from the underside 32 of damaged tissue layer 26 (see FIG. 2), or from undamaged tissue region 28 of specimen 10.

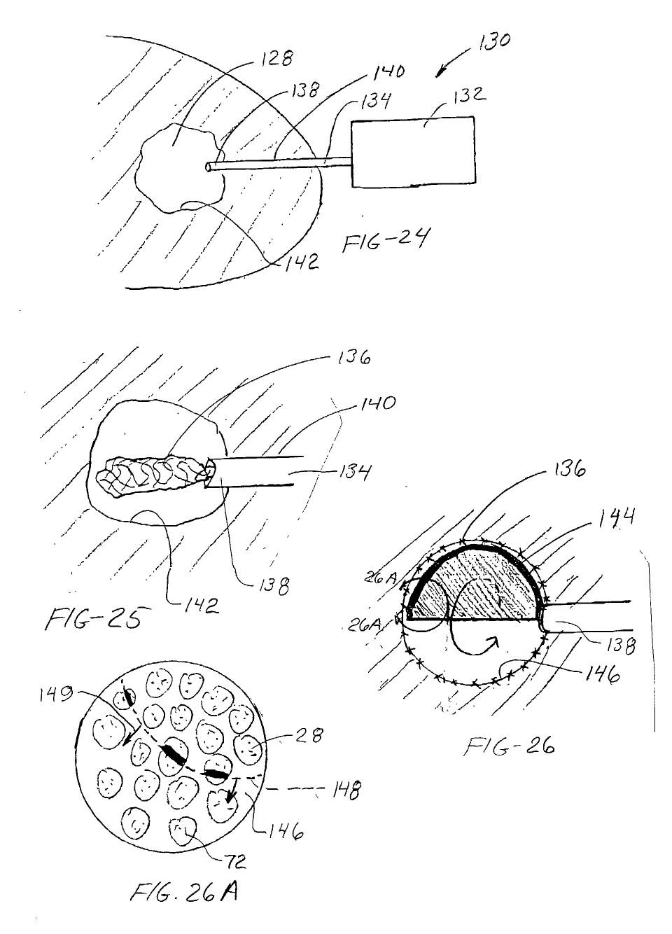

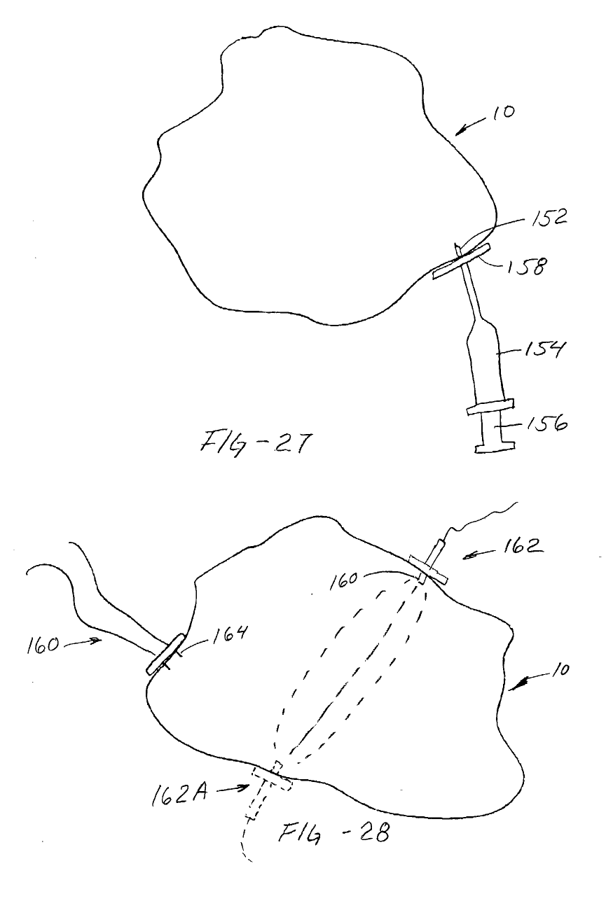

FIGS. 24-26A illustrate obtaining undamaged tissue from an excision void 128, or other cavity, using a void wall tissue sample collection assembly 130. Assembly 130 comprises an operational unit 132 from which an introducer sheath 134 extends. A radially expandable and contractible apertured void wall engagement device 136 is housed within introducer sheath 134 as the open distal end 138 of sheath 134 is passed along the tissue tract 140 and into excision void 128. Engagement device 136 is then extended through open distal end 138 in a retracted state as shown in FIG. 25. Engagement device 136 is preferably a braided structure, truss structure or other structure that has apertures and that can be expanded to press against the void wall 142 of void 128 with sufficient force to cause void wall tissue to extend inwardly through the apertures in device 136; one example of this is shown in FIG. 26A with protruding damaged tissue 72 passing through generally circular apertures. Engagement device may be, for example, mechanically expandable, expandable using fluid pressure or expandable using electrical energy or heat. Assembly 130 also includes a radially expandable, rotatable blade 144 which passes along the inner surface 146 of device 136 when device 136 is in the expanded state of FIG. 26.

FIG. 26A is an enlarged view of a portion of inner surface 146 of engagement device 136 illustrating the path 148 of blade 144 as it passes over surface 146 in the directional arrows 149 thereby severing protruding damaged tissue 72 and leaving behind undamaged tissue regions 28. Blade 144 and device 136 can then be collapsed and withdrawn from excision void 128 and into introducer sheath 134 and introducer sheath may be removed from the patient. Tissue collected within engagement device 136 may be tested. All or only a portion of inner surface 146 may be acted on by blade 144. Assembly 130 could be made with more than one blade 144. Blade 144, or other severing element, may not be a radially expandable element.

It may be desirable to insert a balloon or other expandable element into excision void 128 after the removal of engagement device 136, expand the expandable element against the newly exposed undamaged tissue regions 28, collapse the expandable element, remove the expandable element from the patient, and reexpand the expandable element to provide access to undamaged tissue. The undamaged tissue may be analyzed using Touch Prep or other procedures. This procedure may also be used without first using assembly 130 when excision void 128 was made without significant damage to the tissue at void wall 142.

FIG. 27 illustrates a syringe-type tissue sampling device 150 comprising a needle 152 extending from a barrel 154 housing a plunger 156. A stop 158 is used to control the insertion depth of 152 so that only undamaged tissue is removed for analysis.

Similarly, a probe-like device could be placed to a certain depth inside the tissue sample and then the probe could sample certain characteristics of the tissue (including, but not limited to optical reflectivity/refractivity, impedance, resistivity, conductivity, etc.). This technology and method could then determine the characteristics and subsequent diagnosis of the ‘undamaged’ tissue. FIG. 28 illustrates two such probe-like devices, that is, tissue characteristic analysis devices 160 and 162, device 160 having multiple tissue characteristic analysis probes 164 while device 162 has a single probe 164. Two or more tissue characteristic analysis devices can be used together to measure impedance density or other characteristics that would indicate cancer, or some other condition, and potentially identify the depth of the item of concern. This is illustrated in FIG. 28 with device 162A shown in dashed lines used in conjunction with device 162 for such measurements.

Another aspect of the invention relates to the analysis of the removed tissue using imaging (or other diagnostic) techniques that would ignore tissue that has been damaged by the removal technologies used. The damaged tissue will have different characteristics than non-damaged tissue and these characteristics could be interpreted and programmed into imaging techniques so that the imaging techniques would ignore the tissues that generate these certain characteristics. Such imaging techniques include, but are not limited to MRI, US, PET, CT, X-ray, photo-spectral analysis, electron microscopic analysis, etc. One such device and method using the aforementioned imaging technologies (or other diagnostic modality) could be designed so that undesirable tissue characteristic(s) could be ignored.

When orientation is not critical, one may place the entire sample into a container of, for example, saline, spin out all the liquid, and then evaluate the cells. This would result in a sampling of cells from all over the sample, not just particular places. This is very similar to the Pap technique that is used today. In the Pap “smear” technique, the tissue is swabbed. After that, the swab is rolled onto a slide for evaluation. Only about 10% of the cells from the swab make it to the slide. The newer Pap technique is to place the swab in a bath of saline, remove the liquid from the cells, and then place the cells onto the slide. It is believed that this technique captures about 90% of the cells. This technique might also prove useful for tumorectomy procedures, as it would yield more cells, but would not give orientation. This technique may be accomplished in conjunction with an orientation method. This technique may also be used to see if there are any cancer cells on the surface of the tissue.

Although the foregoing ideas have been described in some detail by way of illustration and example, for purposes of clarity of understanding, it will be obvious that certain changes and modifications may be practiced within the scope of the appended claims. For example, other techniques of tissue removal, such as reciprocating or vibrating mechanical cutting/severing devices or chemical etching procedures, could be used.

Any and all patents, patent applications and printed publications referred to above are incorporated by reference.

What is claimed is:

1. A tissue sample collection assembly, for collecting undamaged cells from a tissue specimen, comprising:
a reference surface placeable against a tissue specimen, the tissue specimen comprising a damaged tissue layer at a margin of the tissue specimen, the damaged tissue layer having a first, expected thickness; and
a tissue-severing device spaced apart from the reference surface by a second distance, said second distance being at most about 40% greater than the first thickness;
whereby said tissue-severing device may be used to separate at least a portion of the damaged tissue layer of the tissue specimen so that undamaged cells may be analyzed.

2. The assembly according to claim 1 wherein said tissue-severing device has a tissue-adhesive surface facing the reference surface so that undamaged cells may contact and adhere to the tissue-adhesive surface.

3. The assembly according to claim 1 wherein said second distance is at most about 20% greater than the first thickness.

4. The assembly according to claim 1 wherein said second distance is at most about 10% greater than the first thickness.

5. The assembly according to claim 1 wherein the tissue-severing device is non-removably mounted to the reference surface.

6. The assembly according to claim 1 wherein the tissue-severing device and the reference surface are fixed relative to one another.

7. The assembly according to claim 1 wherein the tissue-severing device and the reference surface are movable relative to one another.

8. The assembly according to claim 1 further comprising a vacuum source coupleable to the reference surface.

9. The assembly according to claim 8 wherein the tissue-severing device is movable from a first position laterally offset from the reference surface to a second position laterally aligned with the reference surface so to separate said portion of the damaged tissue layer during such movement.

10. The assembly according to claim 1 wherein the tissue-severing device comprises a flat, solid blade.

11. The assembly according to claim 1 wherein the tissue-severing device comprises a translucent or transparent portion, said translucent or transparent portion comprising said tissue-adhesive surface.

12. The assembly according to claim 1 wherein the tissue-adhesive surface comprises a removable, double-adhesive-sided tape.

13. A tissue sample collection assembly, for collecting undamaged cells from a tissue specimen, comprising:
a reference surface placeable against a tissue specimen, the tissue specimen comprising a damaged tissue layer at a margin of the tissue specimen, the damaged tissue layer having a first, expected thickness;
a tissue-severing device spaced apart from the reference surface by a second distance, said second distance being at least as great as the first thickness; and
said tissue-severing device having a tissue-adhesive surface facing the reference surface;
whereby said tissue-severing device may be used to separate at least a portion of the ii damaged tissue layer of the tissue specimen so that undamaged cells may contact and adhere to the tissue-adhesive surface.

14. The assembly according to claim 13 wherein the tissue-severing device is non-removably mounted to the reference surface.

15. The assembly according to claim 13 wherein the tissue-severing device and the reference surface are fixed relative to one another.

16. The assembly according to claim 13 wherein the tissue-severing device and the reference surface are movable relative to one another.

17. The assembly according to claim 13 further comprising a vacuum source coupleable to the reference surface.

18. The assembly according to claim 17 wherein the tissue-severing device is movable from a first position laterally offset from the reference surface to a second position laterally aligned with the reference surface so to separate said portion of the damaged tissue layer during such movement.

19. The assembly according to claim 13 wherein the tissue-severing device comprises a flat, solid blade.

20. The assembly according to claim 13 wherein the tissue-severing device comprises a translucent or transparent portion, said translucent or transparent portion comprising said tissue-adhesive surface.

21. The assembly according to claim 13 wherein the tissue-adhesive surface comprises a removable, double-adhesive-sided tape.

22. A tissue sample collection assembly, for collecting undamaged cells from beneath a damaged tissue layer at a margin of a tissue specimen, the damaged tissue layer having a first thickness, comprising:
an apertured device comprising inner and outer surfaces with apertures passing therebetween;
a tissue-severing device having a tissue-adhesive surface placeable adjacent to the outer surface; and
said apertures sized and shaped so that when the inner surface of the apertured device is pressed against a tissue specimen, portions of an undamaged tissue layer of the tissue specimen pass through the apertures and past the outer surface so that the tissue-severing device can sever the undamaged tissue layer portions from the remainder of the tissue specimen, whereby exposed undamaged tissue may adhere to the tissue-adhesive surface of the tissue-severing device.

23. The assembly according to claim 22 wherein the apertured device comprises a generally tubular braided device.

24. The assembly according to claim 23 wherein the tissue-severing device comprises a curved cutting edge.

25. The assembly according to claim 24 wherein the tissue-severing device is generally tubular.

26. The assembly according to claim 24 wherein the tissue-severing device comprises a resilient, coiled, generally tubular blade.

27. The assembly according to claim 23 wherein the generally tubular braided device contracts radially when placed in tension longitudinally.

28. The assembly according to claim 23 further comprising a tissue-impervious, removable protective layer surrounding the apertured device to help prevent seeding of tissue cells from the tissue specimen when the tissue specimen is removed from a patient along a tissue track of the patient.

29. The assembly according to claim 28 wherein the protective layer comprises a second generally tubular braided device.

30. A method for analyzing undamaged tissue from a margin of a tissue specimen of a type in which the margin comprises a damaged tissue layer overlying undamaged tissue, said damaged tissue layer comprising damaged tissue, said damaged tissue having been damaged during a tissue specimen removal procedure, the method comprising:
determining an expected thickness of damaged tissue;
selecting a chosen thickness of damaged tissue to be removed, said chosen thickness being greater than said expected thickness;
removing at least a portion of the damaged tissue layer to expose a region of the undamaged tissue, said portion of the damaged tissue layer having an inner surface;
analyzing tissue from at least a chosen one of the undamaged tissue region and said inner surface of said portion of the damaged tissue region; and
the selecting step comprising minimizing said chosen thickness to help ensure that the tissue analyzed is close to the margin of the tissue specimen to help with the determination of whether there is tissue of interest at the margin.

31. The method according to claim 30 wherein the removing step comprises incremental removal of layers of damaged tissue to expose said region of undamaged tissue.

32. The method according to claim 30 wherein the removing step comprises:
pressing an apertured device, comprising at least one aperture, and the damaged tissue layer against one another causing damaged tissue to protrude through said aperture; and
separating at least some of the protruding damaged tissue from the remainder of the tissue specimen to expose said region of underlying undamaged tissue.

33. The method according to claim 32 wherein the pressing step is carried out using an apertured device comprising outer and inner surfaces, the separating step comprising pressing the outer surface against the tissue specimen and passing a blade over the inner surface.

34. The method according to claim 33 wherein the blade passing step comprises rotating the blade over the inner surface.

35. The method according to claim 32 wherein the pressing step is carried out using an apertured device comprising a stiff member having a plurality of said apertures.

36. The method according to claim 32 wherein the pressing step is carried out using an apertured device comprising a mesh.

37. The method according to claim 32 wherein the pressing step is carried out using an apertured device comprising a generally tubular braided device.

38. The method according to claim 37 and wherein the pressing step comprises placing the tissue specimen within the generally tubular braided device and applying tension on the generally tubular braided device thereby contracting the generally tubular braided device onto the tissue specimen.

39. The method according to claim 38 wherein the separating step comprises passing a curved blade edge over the braided device.

40. The method according to claim 39 wherein the passing step is carried out using an at least substantially continuous loop, curved blade edge.

41. The method according to claim 39 further comprising adjusting a transverse dimension of the curved blade edge.

42. The method according to claim 41 wherein the transverse dimension adjusting step is carried out using a resilient coiled blade.

43. The method according to claim 32 wherein the pressing step is carried out using an apertured device comprising first and second, generally tubular, braided devices, the first braided device comprising a plurality of said apertures, said second braided device having a tissue-impervious surface to prevent tissue from passing therethrough, said first braided device at least partially housed within the second braided device.

44. The method according to claim 43 wherein the pressing step comprises placing the tissue specimen within the first braided device.

45. The method according to claim 44 wherein the pressing step comprises applying tension on the first braided device thereby contracting the first braided device onto the tissue specimen.

46. The method according to claim 45 further comprising withdrawing the first braided device, and the tissue specimen therein, from within the second braided device.

47. The method according to claim 46 wherein the withdrawing step takes place after the applying tension step.

48. The method according to claim 30 wherein the removing step comprises abrading the damaged tissue layer.

49. The method according to claim 30 wherein the removing step comprises passing a tissue-separating tool over the damaged tissue layer.

50. The method according to claim 49 further comprising selecting a tissue-separating tool according to the expected thickness of the damaged tissue layer.

51. The method according to claim 49 wherein the passing step is carried out using a tissue-separating tool having a plurality of individual tissue-severing edges.

52. The method according to claim 49 wherein the passing step is carried out using a tissue-separating tool having one tissue-severing edge.

53. The method according to claim 49 wherein the passing step is carried out using a vacuum-assisted tissue-separating tool comprising a vacuum surface and a tissue-severing element associated with the vacuum surface.

54. The method according to claim 53 wherein the passing step is carried out using a tissue-separating the tool having a tissue-contacting surface and having said vacuum surface set back from said tissue-contacting surface.

55. The method according to claim 49 wherein the tissue-separating tool passing step comprises adhering tissue from the undamaged tissue region to a tissue sample region of the tissue-separating tool.

56. The method according to claim 55 wherein the adhering step is carried out using an adhesive surface at the tissue sample region.

57. The method according to claim 55 wherein the adhering step is carried out using a removable adhesive surface device at the tissue sample region.

58. The method according to claim 55 wherein the adhering step is carried out using a removable adhesive tape at the tissue sample region.

59. The method according to claim 55 wherein the tissue analyzing step is carried out on the tissue adhering to the tissue sample region.

60. The method according to claim 30 wherein the analyzing step comprises removing a tissue sample from the undamaged tissue region.

61. The method according to claim 60 further comprising:
placing the tissue sample into a liquid to create a tissue sample cell/liquid mixture;
removing liquid from the tissue sample cell/liquid mixture to leave tissue sample cells; and
analyzing the tissue sample cells.

62. The method according to claim 30 wherein the analyzing step comprises at least one of the following: optical reflectivity of the tissue, radiographic analysis, histological analysis, pathologic analysis, MRI analysis, electrical analysis, resistive analysis, impedance analysis, refractive analysis, cell analysis, ultrasound analysis, fine needle aspiration analysis.

63. The method according to claim 30 wherein the removing step comprises:
placing the tissue sample within an open-ended container, said container having an inner wall with a chosen cross-sectional shape, with the tissue specimen adjacent to the inner wall; and
passing a cutting device into the container, said cutting device having a cutting edge positionable a chosen distance inwardly of the inner wall.

64. The method according to claim 63 wherein the tissue specimen placing step is carried out using a container having a cylindrical inner wall.

65. The method according to claim 64 wherein a cutting device passing step is carried out using a cutting device having a circular edge.

66. A method for analyzing undamaged tissue from a margin of a tissue specimen of a type in which the margin comprises a damaged tissue layer overlying undamaged tissue, said damaged tissue layer comprising damaged tissue, said damaged tissue having been damaged during a tissue specimen removal procedure, the method comprising:
removing a tissue sample from the margin of the tissue specimen, the tissue sample comprising damaged tissue and undamaged tissue;
selecting an analysis technique that differentiates between damaged tissue and undamaged tissue in a tissue sample; and
analyzing undamaged tissue from the tissue sample.

67. The method according to claim 66 wherein the analyzing step is carried out using at least one of MRI, US, PET, CT, X-ray, photo-spectral analysis and electron microscopic analysis.

68. A method for analyzing undamaged tissue from a margin of a tissue specimen of a type in which the margin comprises a damaged tissue layer overlying undamaged tissue, said damaged tissue layer comprising damaged tissue, said damaged tissue having been damaged during a tissue specimen removal procedure, the method comprising:
inserting a tissue sampling device into the damaged tissue layer and through damaged tissue to access undamaged tissue;
capturing an undamaged tissue sample using the tissue sampling device;
removing the undamaged tissue sample from the tissue specimen; and
analyzing the undamaged tissue sample.

69. A method for analyzing undamaged tissue from a margin of a tissue specimen of a type in which the margin comprises a damaged tissue layer overlying undamaged tissue, said damaged tissue layer comprising damaged tissue, said damaged tissue having been damaged during a tissue specimen removal procedure, the method comprising:
inserting a tissue characteristic analysis probe into the damaged tissue layer and through damaged tissue to access undamaged tissue; and
analyzing the undamaged tissue for said tissue characteristic using the tissue characteristic analysis probe.

70. The method according to claim 69 wherein the inserting step is carried out by inserting a plurality of analysis probes at spaced-apart positions.

71. The method according to claim 69 wherein the inserting step is carried out by inserting analysis probes at positions on opposite sides of the tissue specimen.

72. A method for obtaining a tissue analysis sample following removal of a tissue specimen through an access track of a patient, the access track opening into a tissue specimen excision void of the patient, comprising:
selecting a sample retrieval structure, movable between a collapsed state an expanded state, the sample retrieval structure having a tissue-adhesive surface;
inserting the sample retrieval structure in a first collapsed state along an access track and into an excision void of a patient;
expanding the sample retrieval structure to an expanded state;
pressing the tissue-adhesive surface against a wall defining the excision void thereby causing tissue from said wall to adhere to the tissue-adhesive surface;
collapsing the sample retrieval structure to a second collapsed state; and
removing the sample retrieval structure, together with tissue adhering to the tissue-adhesive surface, from the patient, whereby said tissue adhering to the tissue-adhesive surface may be analyzed.

73. The method according to claim 72 further comprising removing a layer of tissue from at least a portion of the wall defining the excision void prior to the inserting step so to remove damaged tissue and expose undamaged tissue.

74. The method according to claim 72 wherein the selecting step comprises selecting an inflatable sample retrieval structure having an external surface.

75. The method according to claim 74 wherein the selecting step is carried out with the tissue-adhesive surface covering at least a portion of the external surface.

76. The method according to claim 74 wherein the expanding step is carried out by inflating the sample retrieval structure.

77. The method according to claim 74 wherein the selecting step comprises selecting a compliant, substantially non-elastic balloon as the inflatable sample retrieval structure.
